(19) United States
(12) Patent Application Publication
(10) Pub. No.: US 2003/0118515 A1
(43) Pub. Date: Jun. 26, 2003

(54) Cosmetic composition containing carbon dioxide

(76) Inventors: Robert Jew, San Francisco, CA (US); Jean Jew, Iowa City, IA (US)

Correspondence Address: Supervisor, Patent Prosecution Services, PIPER MARBURY RUDNICK & WOLFE LLP, 1200 Nineteenth Street, N.W., Washington, DC, 20036-2412, US

(21) Appl. No.: 10/024,245

(22) Filed: Dec. 21, 2001

(51) Int. Cl. A61K 9/00

(52) U.S. Cl. 424/47

(57) ABSTRACT

The present invention relates to cosmetic compositions that contain at least 0.1 wt % carbon dioxide in a cosmetically acceptable carrier. Preferably the cosmetic compositions contain from about 0.1 to about 8.0 wt. % carbon dioxide, and from about 92.0 to about 99.9 wt % of a dermatologically acceptable fluorocarbon free carrier. The cosmetic compositions according to the present invention improve the respiratory function of the skin to facilitate and enhance skin treatment and improve the skin's overall appearance. The present invention also relates to a method of increasing the oxygen content in the surface of skin by topically applying the cosmetic compositions of the present invention to skin.

### BACKGROUND OF THE INVENTION

1. Field of the Invention

The present invention relates generally to cosmetic compositions that contain carbon dioxide. In particular, the cosmetic compositions according to the present invention produce desirable skin cosmetic effects due to improved metabolism of skin cells. The present invention further pertains to a method of producing a desirable skin cosmetic effect by applying the cosmetic compositions of the present invention to skin.

2. Discussion of the Background

It is well-known that oxygen is an essential component in the metabolism of skin cells and that oxygen delivery to these cells operates through the hemoglobin complex in the red blood cells. The amount of oxygen that is available for metabolism inside skin cells is a function of cutaneous micro-circulation, respiration, environment, health, age, and physical activity. Oxygen levels in the skin are lower than in any other parts of the body and are known to decrease with age. Oxygen is used by the skin to produce energy to fuel cellular processes such as proliferation and production of extracellular matrix substances, both of which are essential for maintaining healthy, young looking skin. Thus, skin care compositions that increase the level of oxygen in the skin are desired.

It has been hypothesized that skin treatments that incorporate the delivery of molecular oxygen or compressed oxygen gas to the surface of the skin have a beneficial effect on the respiratory function of skin cells. In particular, it is believed that making more oxygen available to the surface of the skin would provide a beneficial cosmetic effect. However, such exogenous treatments of oxygen are dangerous because oxygen is extremely flammable. Thus, oxygen treatments provide manufacturing challenges and an increase in cost. Furthermore, products containing oxygen do not allow a co-presence of agents that are sensitive to oxidation, such as antioxidants or retinoids, because such agents are unstable in the presence of oxygen.

In recent research conducted and published by Komoto et al., it was shown that elevation of subcutaneous tissue PO₂ (oxygen partial pressure) was followed by an elevation of PCO₂ (carbon dioxide partial pressure in the subcutaneous tissue). (Komoto et al., Elevation of tissue PO₂ with improvement of tissue perfusion by topically applied CO₂, Advanced Experimental Medical Biology, 1988; 222: 637-45). In addition, it has been recently demonstrated that vasodilation and increased oxygen utilization (i.e., Bohr effect) results from topical CO₂ application. (Hartmann et al., Effect of carbon dioxide enriched water and fresh water on the cutaneous micrcocirculation and oxygen tension in the skin of the foot, Aniology, April, 1997, 48(4): 337-43).

The present inventors believed that the skin cell's metabolic processes would be better served with treatments that increase cutaneous oxygen elevation, consumption, and utilization. In this regard, the present inventors discovered that when carbon dioxide (CO₂) is mixed with a suitable carrier, and that mixture is applied to the surface of the skin, cutaneous circulation, vasodilation, and increased oxygen utilization occurs in variable specific circumstances.

### SUMMARY OF THE INVENTION

It is an object of the present invention to provide a cosmetic composition that includes carbon dioxide and a suitable carrier.

It is another object of the present invention to provide a cosmetic composition that provides a beneficial effect on the skin, the skin's cells, and the skin's metabolic processes.

It is a further object of the present invention to increase the amount of oxygen present in the skin.

It is yet another object of the present invention to increase and improve oxygen perfusion as a result of combining carbon dioxide in an admixture of cosmetic ingredients.

It is another object of the present invention to provide a method for producing a desirable skin cosmetic effect.

It is a further object of the present invention to provide a method that avoids the use of agents that release oxygen by means of a chemical reaction.

It is yet another object of the present invention to provide a cosmetic composition that is maintained in a pressurized container.

It is another object of the present invention to provide a cosmetic composition that contains a dermatologically acceptable fluorocarbon-free carrier.

It is an advantage of the present invention that oxidative damage to skin cells is avoided by physically releasing carbon dioxide.

Further advantages of the present invention include reduction in skin wrinkles, increased skin hydration, improved appearance of skin, and improved texture of skin.

These and other objects of the present invention are met by a composition of matter for producing a desirable skin cosmetic effect due to the improved respiratory function and/or metabolism of skin cells comprising carbon dioxide and a dermatologically acceptable carrier. Preferably, the cosmetic composition includes about 0.1 to about 8.0 wt % carbon dioxide with about 92.0 to about 99.9 wt % of a dermatologically acceptable fluorocarbon-free carrier.

### DETAILED DESCRIPTION OF THE INVENTION

To achieve the foregoing and other objects, and in accordance with the purpose of the present invention as embodied and broadly described herein, the present invention relates to cosmetic compositions that contain carbon dioxide and a suitable dermatologically acceptable carrier. In particular, the cosmetic compositions includes at least 0.1 wt % carbon dioxide, preferably from about 0.1 to about 8.0 wt % carbon dioxide, and about 92.0 to about 99.9 wt % of a dermatologically acceptable carrier. It is preferred that the carrier is fluorocarbon free. These cosmetic compositions produce desirable skin cosmetic effects due to improved metabolism of skin cells and improved respiratory function of the skin. The present invention further pertains to a method of producing a desirable skin cosmetic effect by applying the cosmetic compositions of the present invention to skin. Desirably, the cosmetic composition is applied to the skin from a pressurized container, such as an aerosol container.

The carbon dioxide utilized in accordance with the present invention is preferably substantially pure, although a gas which is enriched in its carbon dioxide content, and which preferably contains at least a major amount of carbon dioxide and trace amounts or at most minor amounts of other harmless gases may also be used. Air, which contains carbon dioxide, is not the intended source of carbon dioxide for this invention, and more important, is impractical for the present purpose of providing the relatively high carbon dioxide concentration required in the composition of the present invention. Carbon dioxide may be used to provide both the active ingredient of composition as well as a propellant in certain embodiments.

The dermatologically acceptable carrier according to the present invention is not specifically limited, and includes carriers which have been utilized in the cosmetic art and would be easily identifiable by one of skill in the art. Preferably, the carrier used in the compositions of the present invention is also cosmetically acceptable, i.e., is non-irritating and does not otherwise adversely affect the skin. It is even more preferable that the carrier is fluorocarbon-free. The carrier may be in the form of a gas, liquid, or semi-solid. Suitable examples of carriers for use in the present invention include, but are not limited to, creams or lotions such as face creams, body lotions, hair creams or lotions, skin moisture creams, other skin conditioning creams, skin masks, liniments, and shaving creams. An example of a dermatologically acceptable carrier which is not necessarily cosmetically acceptable is petroleum jelly, which may be also be used as a carrier in accordance with the present invention.

The carrier may optionally contain a dermatologically acceptable form of retinoic acid to improve the skin benefits obtained by the cosmetic compositions. Preferably, retinol is incorporated in the cosmetic compositions, as it has been disclosed in medical texts as having better penetration and less irritation than retinoic acid in the topical treatment of photodamaged skin. The carrier may also contain dermatologically acceptable forms of tocopherol, hyaluronic acid, glycoaminoglycans, polyphenols, allium compounds, and essential lipids which contribute to the function and physical health of the skin.

The carrier may also contain cosmetic ingredients which improve skin protection, appearance, moisture, irritation, or texture. Such ingredients include, but are not limited to butylene glycol, propylene glycol, aloe, glycerin, glycerin esters, polyethylene glycol ethers, hyaluronic acid, vitamin K, biotin, octyl palmitate, polysorbates, polyquaterniums, jojoba, calendula, extract, chamomile extract, grape extract, horsetail extract, garlic extract, licorice extract, rosemary extract, thiamin, azelaic acid, lactic acid, caprylic acid, caproic acid, capric acid, linoleic acid, xanthum, kinetin, shea butter, wheat proteins, soy proteins, oat proteins, phospholipids, liposomes, beta glucan, dimethicones, fennel oil, essential oils, essential fatty acids, sunblock/sunscreen (e.g. titanium dioxide, Parson 1789, octocrylene),lecithin, and cetyl esters.

Further, the carrier may contain ingredients that have been cited as potential carriers of oxygen and/or yield oxygen under conditions of chemical biosynthesis such as shark liver oil, olive oil, and squalane. In addition, the cosmetic compositions may also contain preservatives such as parabens (e.g., alkyl esters such as methyl, ethyl, propyl and butyl esters of p-hydroxybenzoic acid).

In a preferred embodiment of the present invention, the carrier includes water and/or organic media such as, for example, ethanol, propanol, isopropanol, glycols, esters and paraffin oil. Thus, as one example of a preferred embodiment, the carrier may include water and ethanol, propanol, isopropanol, glycols, esters or paraffin oil. In another embodiment, water may be absent, or substantially absent (i.e., <10 wt %) from the carrier. In the situation where water is substantially absent from the carrier, the carrier will include organic media such as ethanol, propanol, isopropanol, glycols, esters and parafin oil.

Although carbon dioxide is unlikely to have a deleterious oxidative effect on either the carrier or skin cell constituents due to its physical release in the composition, the cosmetic compositions of the present invention may also contain antioxidants. Suitable examples of antioxidants include BHA (butylated hydroxy anisole), BHT (butylated hydroxy toluene), retinoids, beta-carotene, ubiquinone, propyl gallate, alpha-tocopherol, superoxide dismutase, polyphenols, and ascorbic acid. The carrier may contain from about 0.001 to about 5 wt %, preferably from about 0.01 to about 2 wt % of an antioxidant.

Preferably, the cosmetic compositions are maintained under super-atmospheric pressure prior to use in order to prevent escape of carbon dioxide during storage. In such a situation, the carrier will also contain a propellant. In preferred embodiments, the propellant is present in an amount of from about 1 to about 25 wt %. The propellant may be a pressurized gas, and is preferably one which includes carbon dioxide, nitrous oxide, argon, nitrogen or oxygen, e.g., pressurized air. Where carbon dioxide is used as a propellant in addition to its use as the active ingredient in the cosmetic composition, the intended use and desired effect does not necessarily rely on carbon dioxide as a propellant, as the invention relies on carbon dioxide in the composition and its effect on cutaneous oxygen elevation, consumption, and utilization. Other suitable examples of propellants include propane, butane, isobutane, trichloromonofluoromethane, dichlorodifluoromethane, dichlorotetrafluoromethane, monobromonochlorodifluoromethane, monobrotrichlorodifluoromethane, trichlorotrifluoromethane, tetrafluoromethane, octaflurocyclobutane 1,1,1,2-tetrafluoroethane, 1,1-dichloro-2,2,2-trifluoroethane, 1-chloro-1,1-difluoroethane and dimethyl ether. Examples of the commercially named propellants that may be used include “Dymel 152A”, “Dymel 134a”.

Additionally, either the propellant or the carrier may contain a perflurocarbon liquid or gas to enhance chemical and/or thermal stability while enhancing the solubility of the gases utilized in the composition, including carbon dioxide and oxygen. Fluorocarbons are safe, chemically inert compounds which are capable of dissolving large quantities of carbon dioxide gas. An example of a perfluorocarbon chemical and/or thermal stabilizer used in the present invention is perfluorodecalin or “Flutec PC6”.

In a preferred embodiment, the cosmetic composition of the invention is generated in the form of a foam. The foam provides a superfluous microenvironment of carbon dioxide in contact with the skin. A foam stabilizer may be incorporated in the cosmetic composition, preferably in an amount fo from 0.05 to about 1 wt %. Examples of foam stabilizers include surfactants (e.g., sodium stearate, sodium palmitate, sodium cetyl sulfate), glycerol, proteins, polymeric foam stabilizers such as methylcellulose and polyvinyl alcohol, other foam stabilizers such as saponins, and mixtures thereof. It is to be noted that some commercially available skin lotions may already contain substances that act as foam stabilizers. Thus, it is within the pervue of one of ordinary skill in the art to determine whether a foam stabilizer needs to be added to the cosmetic compositions of the present invention, what type of foam stabilizer would be suitable for use therein, and the optimum proportion thereof.

Having generally described this invention, a further understanding can be obtained by reference to certain specific examples which are provided herein for purposes of illustration only and are not intended to be limiting unless otherwise specified.

### EXAMPLES

### Example 1

In accordance with substantially known industrial procedures, a standard aerosol can of approximately of 130 cc capacity was filled with an admixture of 96 g commercially available skin lotion, 6 g propellant known commercially as “152A”, and more than 1 g of carbon dioxide alternatively measured by 75 lbs per square inch (75 psi). The initial pressure of the can prior to addition of the carbon dioxide was about 50 psi at 70° F.

The contents of the aerosol can were well shaken and the admixture contained therein, which was propelled therefrom in the form of a stabilized foam, was applied to the facial skin around only one eye of the subject. After two weeks of daily morning applications, the depth and definition of the fine lines and wrinkles around the treated eye diminished significantly, albeit without complete disappearance. After four weeks of the daily applications, there was an additional slight improvement in the skin's appearance regarding fine lines, wrinkles and skin texture. After six weeks of daily applications, there was little to no visible improvement to distinguish the results from the results yielded in only four weeks.

After two weeks of discontinued use, the depth and definition of the fine lines and wrinkles around the treated eye returned to some degree, although not to the original condition and appearance of the skin prior to the initial applications.

### Example 2

The procedure set forth in Example 1 was followed using a composition containing 96 g commercially available skin lotion, 6 g propellant known commercially as “Dymel 132a” and more than 1 g of carbon dioxide alternatively measured by 75 lbs per square inch (75 psi). The initial pressure of the can prior to addition of the carbon dioxide was about 50 psi at 70° F. Results similar to Example 1 were obtained.

### Example 3

The procedure set forth in Example 1 was followed using a composition containing 96 g commercially available skin lotion was pressurized with at least 1 g of carbon dioxide measured at 90 psi without any additional propellant, alternatively and distinctively without an additional propellant. The initial pressure of the can prior to addition of the carbon dioxide was about 0.0 psi. at 70° F. Results similar to Example 1 were obtained.

### Example 4

The procedure set forth in Example 1 was followed using a composition containing 96 g commercially available skin lotion was pressurized with at least 1 g of carbon dioxide measured at 100 psi without any additional propellant, alternatively and distinctively without an additional propellant. The initial pressure of the can prior to addition of the carbon dioxide was about 0 psi at 70° F. Results similar to Example 1 were obtained.

The invention of this application as described above both generally, and with regard to specific embodiments. A wide variety of alternatives known to those of skill in the art can be selected within the generic disclosure, and examples are not to be interpreted as limiting, unless specifically so indicated. The invention is not otherwise limited, except for the recitation of the claims set forth below.

What is claimed is:

1. A cosmetic composition for improving the appearance of skin comprising:
from about 0.1 to about 8 wt % carbon dioxide; and
from about 92 to about 99 wt % of a dermatologically acceptable carrier.

2. The cosmetic composition of claim 1, wherein said carrier is free of fluorocarbons.

3. The cosmetic composition of claim 1, wherein said carrier comprises at least one member selected from the group consisting of water, ethanol, propanol, isopropanol, glycols esters and paraffin oil.

4. The cosmetic composition of claim 1, wherein said carrier comprises water and at least one member selected from the group consisting of ethanol, propanol, isopropanol, glycols, esters and paraffin oil.

5. The cosmetic composition of claim 1, wherein said carrier includes a propellant.

6. The cosmetic composition of claim 5, wherein said propellant is present in an amount of from about 1 to about 25 wt %.

7. The cosmetic composition of claim 5, wherein said propellant comprises at least one member selected from the group consisting of carbon dioxide, nitrous oxide, argon, nitrogen, oxygen, propane, butane, isobutane, trichloromonofluoromethane, dichlorodifluoromethane, dichlorotetrafluoromethane, monobromonochlorodifluoromethane, monobrotrichlorodifluoromethane, trichlorotrifluoromethane, tetrafluoromethane, octaflurocyclobutane, 1,1,1,2-tetrafluorethane, 1,1-dichloro-2,2,2-trifluoroethane, 1-chloro-1,1-difluoroethane and dimethyl ether.

8. The cosmetic composition of claim 1, wherein said carrier includes an antioxidant in an amount of from about 0.001 to about 5 wt %.

9. The cosmetic composition of claim 8, wherein said antioxidant is selected from the group consisting of butylated hydroxy anisole, butylated hydroxy toluene, beta-carotene, ubiquinone, propyl gallate, alpha-tocopherol, superoxide dismutase, polyphenols and ascorbic acid.

10. The cosmetic composition of claim 8, wherein said antioxidant is present in an amount of from 0.01 to about 2 wt %.

11. The cosmetic composition of claim 1, wherein said carrier is in the form of a gas, a liquid, or a semi-solid.

12. The cosmetic composition of claim 1, wherein said carrier includes an ingredient for improving skin protection, appearance, moisture, or texture selected from the group consisting of retinol, tocopherol, hyaluronic acid, glycoaminoglycans, polyphenols, allium compounds, essential lipids, butylene glycol, propylene glycol, aloe, glycerin, glycerin esters, polyethylene glycol ethers, octyl palmitate, polysorbates, polyquaterniums, jojoba, calendula, extract, chamomile extract, grape extract, horsetail extract, garlic extract, licorice extract, rosemary extract, thiamin, azelaic acid, lactic acid, caprylic acid, caproic acid, capric acid, linoleic acid, xanthum, kinetin, shea butter, wheat proteins, soy proteins, oat proteins, phospholipids, liposomes, beta carotene, dimethicones, fennel oil, essential oils, essential fatty acids, sun screen, lecithin and cetyl esters.

13. The cosmetic composition of claim 1, wherein said carrier includes an oxygen carrier selected from the group consisting of shark liver oil, olive oil and squalene.

14. The cosmetic composition of claim 1, wherein said carrier includes a foam stabilizer.

15. The cosmetic composition of claim 14, wherein said foam stabilizer is present in an amount of from about 0.05 to about 1.0 wt %.

16. The cosmetic composition of claim 14, wherein said foam stabilizer is selected from the group consisting of sodium stearate, sodium palmitate, sodium cetyl sulfate, glycerol, proteins, methylcellulose, polyvinyl alcohol, saponins and mixtures thereof.

17. A method of improving the appearance of skin comprising:
topically applying the cosmetic composition of claim 1 to skin.

18. The method of claim 17, wherein said carrier is free of fluorocarbons.

19. The method of claim 17, wherein said carrier includes at least one member selected from the group consisting of a propellant, a moisturizer, a preservative, an antioxidant, a perfluorocarbon and a foam stabilizer.

20. The method of claim 17, wherein said carrier comprises at least one member selected from the group consisting of water, ethanol, propanol, isopropanol, glycols esters and paraffin oil.
